(19) United States
(12) Patent Application Publication
(10) Pub. No.: US 2015/0157714 A1
(43) Pub. Date: Jun. 11, 2015

(54) IMPLEMENTATION OF NON-PATHOGENIC BIOLUMINESCENT BACTERIA AS A SUB-MICRON LIGHT EMITTING SOURCE FOR PHOTO-DYNAMIC THERAPY IN THE LUNGS

(71) Applicant: Nomir Medical Technologies, Inc., Woodmere, NY (US)

(72) Inventor: Eric BORNSTEIN, Woodmere, NY (US)

(21) Appl. No.: 14/567,367

(22) Filed: Dec. 11, 2014

Related U.S. Application Data
(60) Provisional application No. 61/914,620, filed on Dec. 11, 2013.

(51) Int. Cl. A61K 41/00; A61N 5/06; A61K 47/46

(52) U.S. Cl. CPC A61K 41/0057; A61K 47/46; A61N 5/062; A61N 2005/0656

(57) ABSTRACT

Systems and methods for treating a pathogenic bacterial infection are provided. The treatment may include introducing bioluminescent bacteria into a lung of a human or animal subject, and performing photodynamic therapy on the lung using light emitted from the bioluminescent bacteria.

### CROSS REFERENCE TO RELATED APPLICATIONS

This application claims the benefit of priority to U.S. Provisional Patent Application No. 61/914,620 entitled “Implementation of non-pathogenic bioluminescent bacteria as a micro-light-emitting source for photo-dynamic therapy” filed Dec. 11, 2013, the entire contents of which are hereby incorporated by reference.

### BACKGROUND

Due to the continuing worldwide escalation of antimicrobial resistance, exploration of new anti-infective approaches has become important in order to find alternative treatments to which resistant microorganisms would be unable to marshal resistance.

Treatment of pulmonary infections with pathogens such as Pseudomonas aeruginosa and Mycobacterium tuberculosis are presently hindered because of extended antibiotic regimens and the emergence of almost complete drug resistance in these and other pathogens. Alternatives and novel therapies to assist or replace standard anti-mycobacterial and anti-pseudomonal agents in infected patients are therefore greatly needed to treat these pulmonary infections.

The lung alveoli contain collagen and elastic fibers that stretch as they fill with air during inhalation, and relax during exhalation to expel the carbon dioxide, as the alveoli are the sites of gas exchange with the blood. A representative set of human lungs contain about 700 million alveoli, which represents about 70 m² of surface area (about 750 sq. feet). Each alveolus is enveloped in a thin fabric of capillaries that cover about 70% of its area. An adult alveolus has an average diameter of 200 microns, which increases in diameter during the inhalation phase.

Photodynamic therapy (PDT) has been discussed in recent years as a novel therapy for treating multidrug resistant pulmonary infections, as it is potentially a way of directly attacking the affecting pathogens with photosensitizers and light in these small sequestered areas. The overwhelming problem however, in a sequestered large surface area such as the lung alveoli, is the lack of a device or mechanism to get a micron-scale light source into the 700 million pulmonary alveoli (200 microns diameter) that are isolated at the terminal ends of the respiratory tree.

A list of publications referenced in this disclosure follows, each of which are incorporated by reference for the relevant portions of their respective disclosures.

### SUMMARY

Various embodiments improve photodynamic therapy (PDT) technology by developing and using a light source that is around one micron in size.

PDT utilizes non-toxic dyes or photosensitizers combined with low intensity visible light, that in the company of oxygen will produce reactive oxygen species (ROS). It is well known that Gram negative bacteria are resistant to many neutral or anionic photosensitizers that will be absorbed only by gram positive species. These photosensitizers will, when stimulated with the correct wavelengths, cause phototoxicity in Gram positive bacterial species.

For gram negative species, photosensitizers utilizing a cationic charge, or utilizing other chemicals (such as lipopolysaccharide permeabilizers) that specifically increase the permeability of the outer membrane in Gm-species, will intensify the absorption in, and effectiveness against these microbes. Most experimental evidence shows that multi-drug resistant bacteria can be eliminated with PDT in the same manner as non-resistant strains. Treatment of infections with PDT also requires absorption selectivity of photosensitizers for the bacteria over healthy host cells.

Therefore, the various embodiments provide live micron-range light sources by developing non-pathogenic, genetically engineered bioluminescent bacteria that would illuminate otherwise impossible to treat localized infections of the lung.

### BRIEF DESCRIPTION OF THE DRAWINGS

The accompanying drawings, which are incorporated herein and constitute part of this specification, illustrate example aspects of the invention, and together with the general description given above and the detailed description given below, serve to explain the features of the invention.

FIG. 1 is a graph of relative emission as a function of wavelength for each of three chemiluminescent luciferases to provide their respective spectral curves.

FIG. 2 is a representative illustration of bioluminescent bacteria in the alveoli of the human lungs.

FIG. 3 is a magnified representative illustration of bioluminescent bacteria in the alveoli of the human lungs, and photosensitizer in proximity to a bacterial pathogen according to an example embodiment.

FIG. 4 is a representative illustration of a pathogenic bacterial infection and biofilm formation in the lung of a cystic fibrosis patient.

FIG. 5 is a representative illustration of a pathogenic bacterial infection, biofilm formation, photosensitizer and bioluminescent bacteria in the lung of a cystic fibrosis patient according to an example embodiment.

FIG. 6 is a representative illustration of a pathogenic bacterial infection and biofilm formation in the lung of a cystic fibrosis patient following the bioluminescent bacterial therapy shown in FIG. 5.

FIG. 7 is a representative illustration of a nebulizer for delivering micron-scale bioluminescent bacteria to the alveoli according to various embodiments.

### DETAILED DESCRIPTION

The various embodiments rely on the utilization of one or more live, micron-scale (e.g., a few microns or less in length, such as 0.5-2 microns long) light source. Examples of such micron-scale light sources may include various genetically engineered non-pathogenic bioluminescent bacteria. These modified organisms may be used to access previously non-accessible areas in the human body (such as the alveoli of the human lungs) and act as the illumination source for photodynamic antimicrobial therapy.

Photodynamic therapy (PDT) is a medical procedure that has three parts: (1) A photosensitizer; (2) visible light of the correct wavelength to interact with and excite a photosensitizer; and (3) molecular oxygen. While no one of these parts is individually toxic to pathogenic microorganisms, when they are combined in PDT, they will produce a photochemical reaction that will generate extremely reactive oxygen species (ROS) via the transference of light energy (electron volts—[eV]) to molecular oxygen (through the photosensitizer), and thereby damage or kill the pathogens that have taken up the photosensitizer.

In an example study using antimicrobial PDT, SP Tseng et al. demonstrated that 60 different multi-drug resistant Pseudomonas aeruginosa (Gram −) isolates could be significantly reduced with PDT utilizing toluidine Blue. (S P Tseng et al., Toluidine blue O photodynamic inactivation on multidrug-resistant Pseudomonas aeruginosa. Lasers Surg Med. 2009 July; 41(5): 391-7). In another example study, K J Hajim et al. demonstrated that the phenothiazinium dye Toluidine Blue O could eradicate methicillin-resistant Staphylococcus aureus (MRSA) with 632.8 nm light. (K J Hajim et al., Laser light combined with a photosensitizer may eliminate methicillin-resistant strains of Staphylococcus aureus. Lasers Med Sci. 2010 September; 25(5): 743-8).

In another example study, T Dai et al. demonstrated almost a 1,000-fold reduction in MRSA utilizing a photosensitizer conjugate of polyethylenimine and chlorin (e6) with light. (T Dai et al. Photodynamic therapy for methicillin-resistant Staphylococcus aureus infection in a mouse skin abrasion model. Lasers Surg Med. 2010 January; 42(1): 38-44). In another example study, B Xing et al. utilized vancomycin-porphyrin conjugates to eradicate vancomycin-resistant Enterococci. (B Xing, et al., Molecular interactions between glycopeptide vancomycin and bacterial cell wall peptide analogues. Chemistry. 2011 Dec. 9; 17(50): 14170-7). These studies are each incorporated by reference herein for the relevant portions of their respective disclosures.

Photosensitizers that absorb visible light have absorption peaks starting approximately in the violet range from 410 nm (chlorin e6) to the far red 773 nm (Si(IV)-naphthocyanine) Light generated at longer wavelengths (near-infrared) does not have enough energy (electron volts/photon) to initiate a photochemical interaction and generate ROS. Visible wavelengths of light can be genetically engineered (programmed through direct manipulation of an organism's genome) into non-pathogenic bacteria, and then have the bioluminescent bacteria delivered to desired areas of treatment with photosensitizers, such as human lung alveoli.

A major limiting factor in successful PDT has always been how to deliver visible light to the desired treatment area. If the desired treatment area is smaller than a few microns, and sequestered in such a way as to make direct illumination impossible with mechanical means (i.e. such as fiber optics or projected energy transmission through tissues), then treatment is impossible. This problem has always kept PDT as a localized treatment (i.e. for wounds), as different researchers have tried to invent many diverse mechanisms of improved optical energy delivery, and improved target localization in inaccessible areas of the human body.

Further, previous delivery devices such as fiber optics, diffusing tip, fiber bundles, implantable light-emitting diodes, or direct illumination have failed to adequately deliver light in these inaccessible areas in the past. Two examples of prior art attempts to project light into the lung alveoli to treat pathogens are given below.

Since Multidrug-resistant Pseudomonas aeruginosa and Burkholderia cepacia (the main causes of morbidity and mortality in cystic fibrosis (CF) patients) have shown susceptibility to methylene blue (MB) and 635 nm light in the past, Cassidy et al. performed a study using MB solution nebulization in an ex vivo and in vitro lung model, where droplets were measured at 4.40 microns. (C M Cassidy et al., Drug and light delivery strategies for photodynamic antimicrobial chemotherapy (PACT) of pulmonary pathogens: a pilot study. Photodiagnosis Photodyn Ther. 2011 March; 8(1): 1-6.) Cassidy tested a fibre-optic light delivery device coupled He—Ne laser, and found that only up to 11% of the total light dose penetrated through full thickness pulmonary parenchymal tissue, to the inside of the lungs. Nakonechny et al. tested liposome-encapsulated methylene blue and toluidine blue, on bacteria under excitation chemiluminescence, with liposome-enclosed luminol (a versatile chemical that exhibits chemiluminescence), in an attempt to eliminate the necessity for an external light source. (F Nakonechny et al., New techniques in antimicrobial photodynamic therapy: Scope of application and overcoming drug resistance in nosocomial infections. In: Méndez-Vilas A (ed.) SCIENCE AGAINST MICROBIAL PATHOGENS: COMMUNICATING CURRENT RESEARCH AND TECHNOLOGICAL ADVANCES. Volume 1. Formatex Research Center Publisher; Badajoz, Spain: 2011. pp. 684-691.)

In view of the limitations of prior PDT therapy, the various embodiments provide micron-scale light sources, such as non-pathogenic bioluminescent bacteria, to increase the potential applications of PDT against deeply sequestered resistant microbial pathogens. In this manner, the clinical applications of PDT may be expanded to include therapeutic light delivery to small pulmonary areas in the human body that were previously inaccessible by conventional mechanical light sources.

In particular, since all bioluminescent reaction pathways discovered to date require oxygen as a cofactor, the lung may provide a beneficial site for the use of the various embodiments. In various embodiments, after an appropriate temporal period of photosensitizer delivery, live micron-scale bioluminescent bacteria may be administered into the infected area of the lung to convey an adequate dose of photon energy (of the corresponding wavelength of light to the photosensitizer) to the pathogenic bacteria that have absorbed the photosensitizer.

In some embodiments, the bioluminescent non-pathogenic bacteria may be genetically engineered for specific functionality as a micron-scale light-emitting source for pulmonary and other types of photodynamic therapy.

Systems and methods of the various embodiments treat a pathogenic infection in a human or animal subject by performing photodynamic therapy on a lung of the subject. In some embodiment methods, performing the photodynamic therapy may introducing a photosensitizer into the lung of the subject, and introducing bioluminescent bacteria into the lung of the subject, in which treatment of the pathogenic infection may be initiated by light emitted from the bioluminescent bacteria. In some embodiment methods, the light emitted from the bioluminescent bacteria may activate or potentiate the photosensitizer.

In some embodiment methods, the light emitted from the bioluminescent bacteria is emitted in a selected wavelength range. In some embodiment methods, the bioluminescent bacteria may be compatible with the commensal microbiome of the lung. In some embodiment methods, the photosensitizer may include a dye. In some embodiment methods, treating the pathogenic infection may include treating one or more pathogenic bacterial species. Embodiment methods may further include administering an antibiotic agent after performing the photodynamic therapy, in which the antibiotic agent is capable of killing weakened pathogenic bacteria. In some embodiment methods, treating the pathogenic infection may include treating one or more pathogenic fungal species. Embodiment methods may further include administering an antifungal agent after performing the photodynamic therapy, in which the antifungal agent is capable of killing weakened pathogenic fungi. In some embodiment methods, introducing the bioluminescent bacteria into the lung of the subject may involve introducing a phototherapeutically effective concentration of bioluminescent microorganisms throughout a substantial portion (e.g., an area of at least about 10-70 m²) of an interior surface area of the lung.

Devices according to the various embodiments may include a reservoir containing bioluminescent bacteria, and an introducer configured to introduce the bioluminescent bacteria into a lung of a human or animal subject in an amount sufficient to perform photodynamic therapy on the lung using light emitted from the bioluminescent bacteria.

Embodiment devices may also include a reservoir containing a photosensitizer. In some embodiment devices, the introducer may be further configured to introduce the therapeutic agent to the lung, and the therapeutic agent may be configured to be activated or potentiated by light emitted from the bioluminescent bacteria.

In some embodiment devices, the introducer may include an inhaler device. In some embodiment devices, the photosensitizer may be preferentially absorbed by a target pathogen over healthy lung tissue. In some embodiment devices, the photosensitizer may maximally absorb light at wavelengths in the visible spectral range corresponding to of the light emitted from the bioluminescent bacteria.

In some embodiment devices, the introducer may be configured to introduce the bioluminescent microorganism into the lung after expiration of a predetermined first time period following the introduction of the photosensitizer. Embodiment devices may be further configured to administer one or more antibiotic compositions to the subject after expiration of a predetermined second time period following the introduction of the photosensitizer to the lung. In some embodiment devices, the predetermined second period of time may be sufficient to allow activation of the photosensitizer. In some embodiment devices, the photodynamic therapy may weaken the target pathogen, which may then be killed by the antibiotic composition. In some embodiment devices, the target pathogen may include one or more species of fungi. Embodiment devices may be further configured to administer one or more antifungal compositions to the subject after expiration of the predetermined second time period following the introduction of the photosensitizer to the lung. In some embodiment devices, the photodynamic therapy may weaken the target pathogen, which may then be killed by an antifungal composition.

### Lung Microbiome

The commensal (non-pathogenic) lung microbiome includes diverse communities of bacteria, fungi and viruses found on the mucus layer and the epithelial surfaces of the alveoli. The bacterial part of the lung microbiome includes almost 140 distinct families, and generally includes nine central bacterial genera: Prevotella, Sphingomonas, Pseudomonas, Acinetobacter, Fusobacterium, Megasphaera, Veillonella, Staphylococcus, and Streptococcus. Typically, Prevotella, Mesorhizobium, Microbacterium, Micrococcus, Veillonella, Rhizobium, Stenotrophomonas, and Lactococcus present mainly in healthy individuals. Therefore, the various embodiments may employ a genetically engineered Bioluminescent single or multiple representative species in one or more of these or other non-pathogenic genera, providing a micron-scale light-emitting source for pulmonary PDT. Specifically, an embodiment bioluminescent micron-scale light-emitting bacteria, or colony of bacteria, may penetrate into the very small and deep areas of the alveoli to activate PDT to weaken or kill harmful bacteria. Examples of harmful bacteria that may infect the lung microbiome and serve as potential targets of pulmonary PDT generally include, but are not limited to, Moraxella catarrhalis, Haemophilus influenzae, Streptococcus pneumoniae, Pseudomonas aeruginosa, and Mycobacterium tuberculosis.

### Cystic Fibrosis

In Cystic Fibrosis (CF) patients, many mechanisms take place that may cause different degrees of lung disease. Specifically, there is increasing difficulty for CF patients to maintain normal respiratory tract mucociliary clearance, which may cause long term infections of multidrug resistant bacteria (e.g., Pseudomonas species, Haemophilus influenzae, Staphylococcus species, etc.). Such chronic lung infections and concomitant inflammation may cause advanced lung disease in CF patients, and are the foremost causes of death associated with CF. The medical community has customarily studied chronic lung infections with the pathogenic bacteria Pseudomonas aeruginosa, Staphylococcus aureus, and Burkholderia cepacia, and the concomitant biofilms in these patients. Along with these species, many investigations have recently recognized a very complex microbial ecology in the lungs of CF patients. For instance, A M Guss et al. examined the sputa from CF patients and identified more than 60 bacterial genera through cloning, culturing, and pyrosequencing techniques. (Guss et al., Phylogenetic and metabolic diversity of bacteria associated with cystic fibrosis. ISME J. 2011 January; 5(1): 20-9.) Further, in other areas of medicine PDT has also shown significant promise in reducing biofilms, biofilm formation, and colony counts within biofilms, which is generally a significant problem in CF patients with respect to various pathogens (e.g., Pseudomonas species).

### Tuberculosis

New infections with Mycobacterium tuberculosis occur in about 1% of the world population each year. In 2007, there were an estimated 13.7 million chronic active tuberculosis (TB) cases around the globe, and in 2010 there were an estimated 9 million new cases of TB and 1.5 million associated deaths, primarily in developing countries. Hence, TB continues to be a tremendous global health problem. In 2012, an estimated 8.6 million people developed TB and approximately 1.3 million died from TB, including over 250,000 deaths from opportunistic TB in patients who are human immunodeficiency syndrome (HIV) positive. Progress toward targets for diagnosis and treatment of multidrug-resistant TB (MDR-TB) is far off track. Worldwide and in most countries with a high burden of MDR-TB, less than 25% of the people estimated to have MDR-TB were detected in 2012.

Some previous research has been aimed at treating Mycobacterium or Pseudomonas species using PDT. These studies include the following, all of which are incorporated by reference for the relevant portions of their respective disclosures: K O'Riordan et al., Photoinactivation of Mycobacteria in vitro and in a new murine model of localized Mycobacterium bovis BCG-induced granulomatous infection. Antimicrob Agents Chemother. 2006 May; 50(5): 1828-34; A Hasebe et al., Mycoplasma Removal from Cell Culture Using Antimicrobial Photodynamic Therapy. Photomed Laser Surg. 2013 March; 31(3): 125-131; N Topaloglu et al., Antimicrobial photodynamic therapy of resistant bacterial strains by indocyanine green and 809-nm diode laser. Photomed Laser Surg. 2013 April; 31(4): 155-62; N Sung et al., Inactivation of multidrug resistant (MDR)- and extensively drug resistant (XDR)-Mycobacterium tuberculosis by photodynamic therapy. Photodiagnosis Photodyn Ther. 2013 December; 10(4): 694-702; and M H Shih et al., Repetitive methylene blue-mediated photo-antimicrobial chemotherapy changes the susceptibility and expression of the outer membrane proteins of Pseudomonas aeruginosa. Photodiagnosis Photodyn Ther. 2013 December; 10(4): 664-71.

However, the previous research does not contemplate or involve a micron-scale light source that could successfully inhabit all or a substantial portion of the 700 million alveoli (each with an average diameter of 200 microns) and the 70 m² of surface area in the lungs necessary to accomplish therapy according to the various embodiments.

### Bioluminescent Bacteria for PDT

The term “bioluminescence” as used herein refers to the production and emission of light by a living organism, which is broadly dispersed in nature, arising in very dissimilar organisms (e.g., bacteria, fungi, insects, fish, squid, etc.). Bioluminescence is a form of chemiluminescence, which refers to the emission of light as the result of a chemical reaction. Organisms that are capable of bioluminescence generally produce a light-emitting pigment (e.g., luciferin) and an enzyme (e.g., luciferase). The luciferin may be any substrate of the luciferase, such as a reduced flavin mononucleotide (FMN) (e.g., FMNH₂) which is known to be a substrate of various bacterial luciferases. Light may be created as a result of enzymatic oxidation of the reduced FMN by bacterial luciferase. A general formula for the oxidation of this luciferin in bacteria is shown in Equation 1:

FMNH₂+O₂+RCHO→FMN+RCOOH+H₂O+hv (Equation 1).

Bacterial luciferase reduces molecular oxygen from the surrounding environment, while oxidizing the reduced FMN (FMNH₂), and oxidizing a long-chain aliphatic aldehyde to an aliphatic carboxylic acid. The reaction forms a series of FMN intermediates, the last of which is an excited hydroxyflavin intermediate. Light is emitted when the excited state of the hydroxyflavin intermediate drops to the ground state, decomposing into the FMN and H₂O. FMN may then be reduced in the presence of FMN reductase and NADH to regenerate the luciferin (e.g., FMNH₂).

Bacterial luciferases may be found in the bacterium Photorhabdus luminescens and marine bacteria from the genera Vibrio and Photobacterium. Bacterial luciferases are encoded by the genes luxAB that form an operon (luxCDABE) together with three additional genes (luxCDE) whose products synthesize the long-chain aldehyde used in Equation 1. An advantage of bacterial luciferase is that it does not require exogenous addition of a luciferin (i.e., substrate), since bacteria have the ability to express the biosynthetic enzymes for substrate synthesis (unlike firefly luciferase).

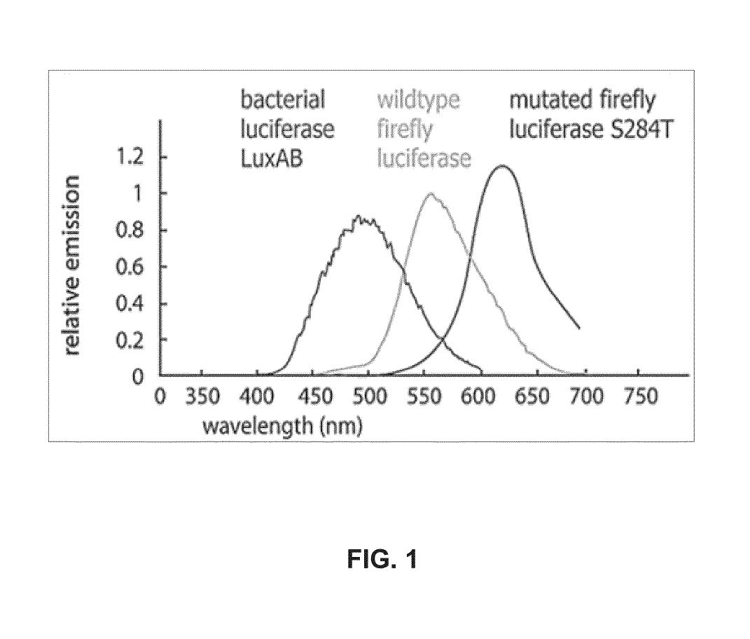

FIG. 1 illustrates representative spectral curves for light produced by a bacterial luciferase, by the wildtype firefly luciferase, and by mutated firefly luciferase S284T. As shown in FIG. 1, bioluminescent bacteria (i.e., having a bacterial luciferase) produce light having a spectral curve that starts at wavelengths of about 430 nm and runs to wavelengths of about 600 nm, with a spectral peak at about 490 nm (light blue in the visible range). Wild-type Firefly luciferase (FFluc) from Photinus pyralis, which uses D-luciferin (a benzothiazole) as substrate, is dependent on ATP, produces light having a spectral curve that starts at wavelengths of about 460 nm and runs to wavelengths of about 680 nm, with a spectral peak at about 557 nm (light green in the visible range). Mutated Firefly luciferase S284T produces light having a spectral curve that starts at wavelengths of about 520 nm and runs to wavelengths of about 720 nm, with a spectral peak at about 630 nm (light red in the visible range).

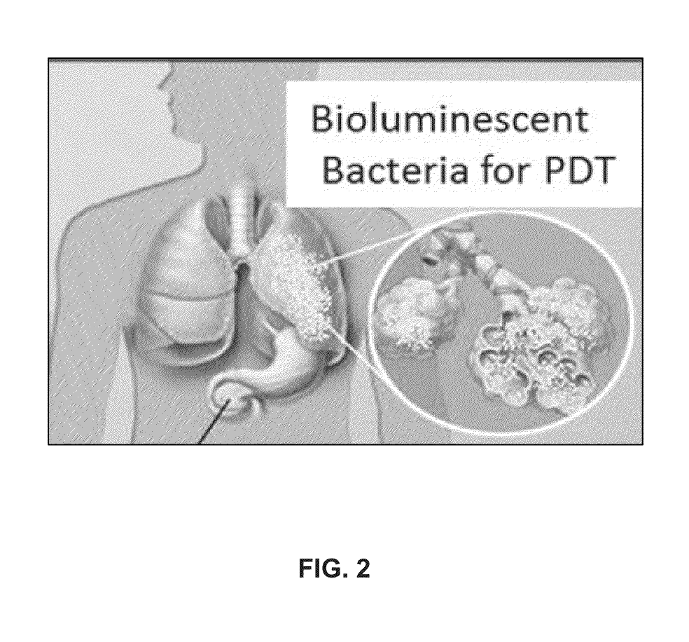

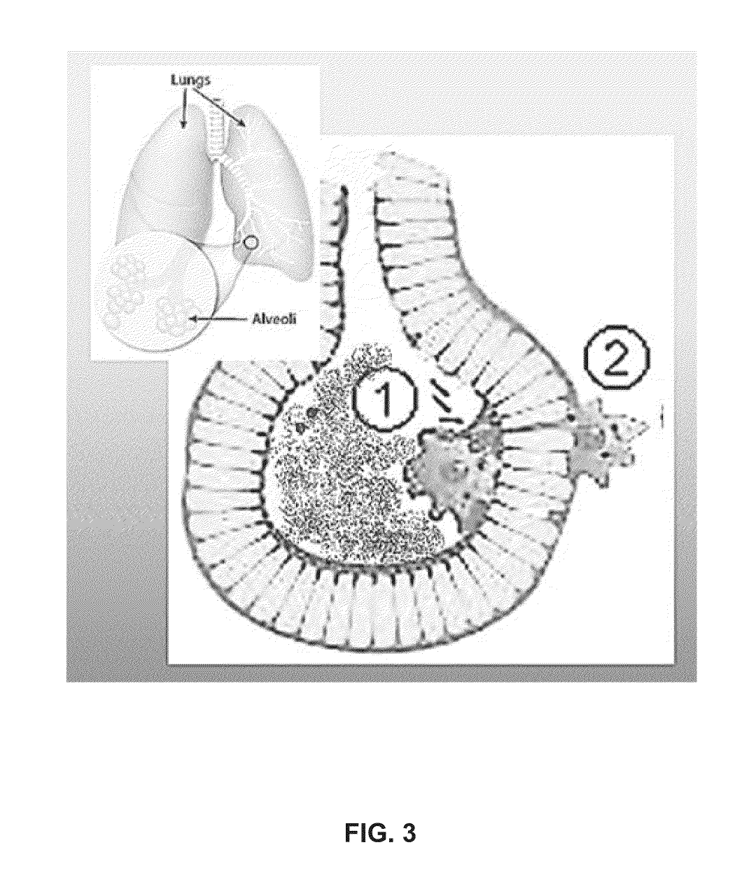

Significant benefits of light-based antimicrobial treatments may include their capacity to eradicate MDR microbes, and the ability to treat areas locally instead of systemically. In various embodiments, the photosensitizer utilized in a PDT treatment may need to demonstrate a high degree of discrimination between target bacteria and normal tissues, especially in areas such as the lungs. Various suitable photosensitizers may be selected that are not harmful until the treatment illumination is applied. FIG. 2 illustrates example lungs containing bioluminescent bacteria for PDT treatment in the alveoli, while FIG. 3 illustrates a magnified view of alveoli containing bioluminescent bacteria (1) and example pathogenic bacteria (2) targeted by a photosensitizer.

Due to the optical absorption characteristics of human tissues, researchers have been attempting to isolate and create photosensitizers that have wavelength absorption spectra in ranges greater than 600. The intensity of light (watts per meter squared (W/m²)) is reduced when passing through tissues, with approximately a 10-fold loss of photon intensity per each cm of tissue depth. When dealing with the necessity of visible light for PDT, hemoglobin is the primary chromophore in human tissues. Since hemoglobin absorbs light strongly within in the blue and green parts of the visible spectrum, blue light and green light are typically poor penetrators of human tissue. In contrast, since absorption of light at wavelengths longer than 600 nm is reduced in human tissue, transmission of orange and red visible light is typically stronger through several centimeters of tissue.

However, photosensitizers suitable for use in the various embodiments beneficially avoid such requirements. That is, in the PDT of the various embodiments, bioluminescent bacteria provide micron-scale light sources in the immediate proximity of (i.e. next to) the pathogenic bacteria in the lung alveoli. Therefore, production of wavelengths in the red visible light range for greater tissue penetration is not necessary, as the emitted light does not need to transmit through any depth of tissue to interact with the photosensitizer. As such, the various embodiments may include photosensitizers that absorb visible light having wavelengths anywhere within bacterial luciferase's spectral range (blue-yellow/orange) from about 430 nm to about 600 nm.

In various embodiments, genetic constructs for engineering the bacteria may be customized for desired wavelengths of light. Specifically, bacteria may be optimized to achieve the maximum possible light expression levels without causing toxicity or adverse effects on the bacteria. Light expression level factors can often be controlled by increasing the levels of reporter gene expression. For example, N Andreu et al. cloned extra promoter genes in front of luxCDE to increase substrate synthesis resulted in a six-fold higher light signal in Mycobacterium smegmatis. (N Andreu et al., Optimisation of bioluminescent reporters for use with mycobacteria. PLoS One. 2010 May 24; 5(5): e10777.) In some embodiments, in order to produce light in the orange and red visible light spectrum, wild-type firefly luciferase or mutated firefly luciferase may be used, with the addition of substrate for the chemiluminescent reaction.

There has also been some early work done on bioluminescent flux (i.e., the amount of energy transferred in the form of photons at a certain distance from the source) in biofilms embedded with bioluminescent bacteria, such as K Blouin, Characterization of a Novel Bacterial Transducer Based on Genetically Engineered Bioluminescence (Febuary 1994) (M.A.Sc. thesis, University of British Columbia), available at https://circle.ubc.ca/bitstream/handle/2429/4965/ubc_{—}1994-0136.pdf?sequence=1.

Vivo Reporter Systems for Gene Expression and Biosensor Applications Based on luxAB Luciferase Genes. Appl Environ Microbiol. 1996 June; 62(6): 2013-21, which is incorporated by reference for the relevant portions of its discourse. As most of these species in the alveoli produce and live in a biofilm, a necessary flux of photons would be emitted to initiate a photochemical reaction in the alveoli.

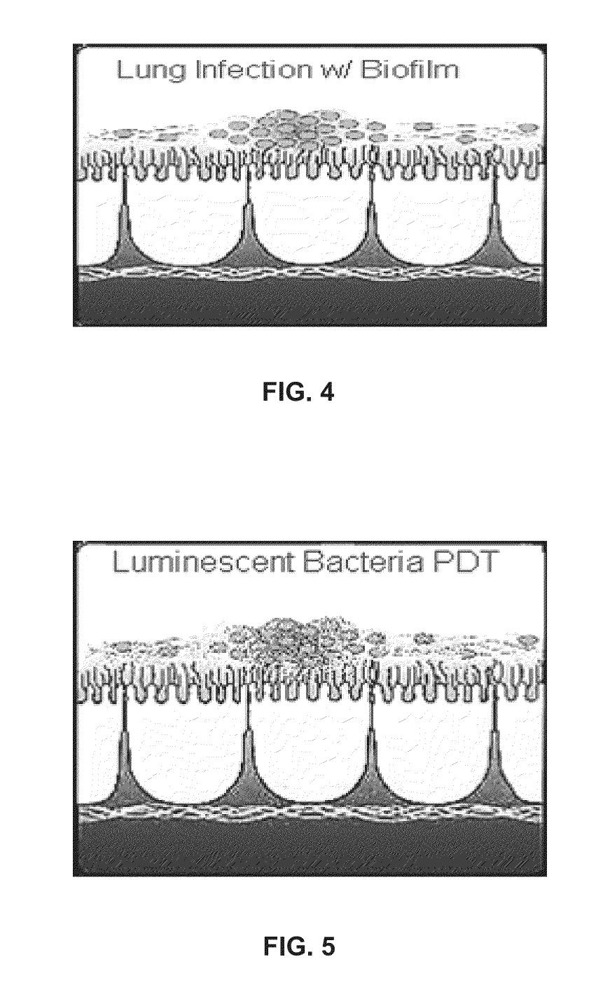

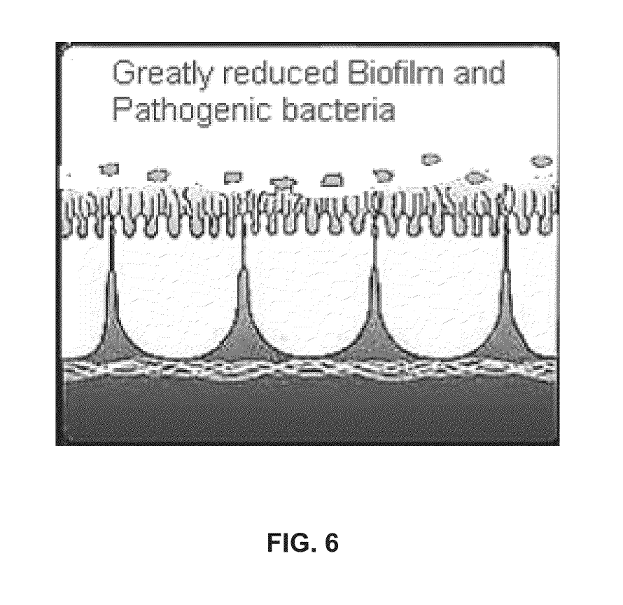

FIG. 4 illustrates a representative pathogenic bacterial infection with a biofilm in, for example, a cystic fibrosis patient's lung, while FIG. 5 illustrates the representative pathogenic infection and biofilm to which a photosensitizer and luminescent bacteria for PDT treatment has been added. As shown in FIG. 6, which illustrates the representative lung following the luminescent bacteria PDT treatment, the biofilm and pathogenic bacterial infection may be greatly reduced.

### Photosensitizers

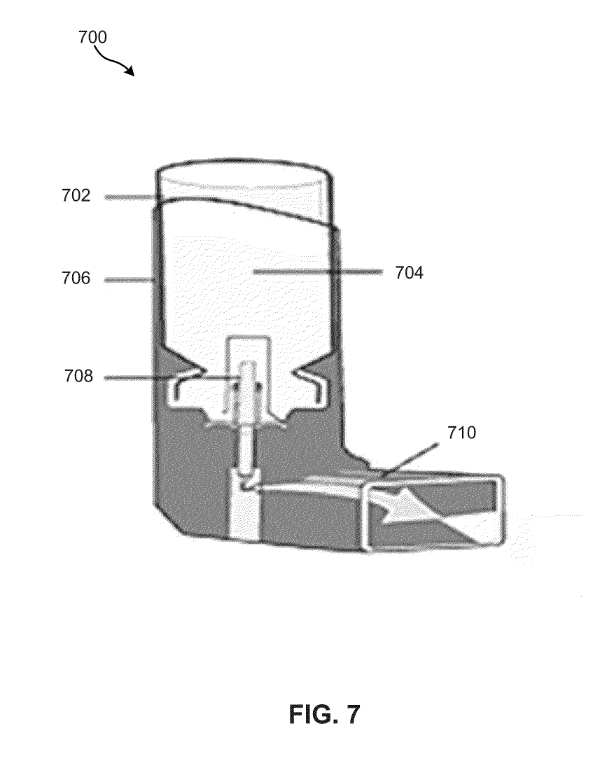

Generally, photosensitizers suitable for use in the various embodiments may have some selectivity for bacterial cells over mammalian cells because they will be administered topically to the lungs instead of through systemic delivery. An example of such topical administration may be performed using a nebulizer. FIG. 7 illustrates components of an example nebulizer 700 that may be used in the various embodiments. Nebulizer 700 may include a canister 702 for containing bioluminescent bacteria 704 for PDT treatment. The canister 702 may be positioned into a plastic holder 706, and coupled to a metering valve 708 for regulating the level of bioluminescent bacteria 704 that is remaining or administered. The patient may receive an easily-inhalable aerosol mist with the bioluminescent bacteria 704 through a mouthpiece 710.

The selectivity for bacterial cells may be enhanced based on the fact that most photosensitizer compounds are designed to be cationic, which allows them to bind to bacterial membranes which are anionic. This would happen preferentially to human cells which are zwitterions or essentially neutral. This difference in membrane binding affinity of photosensitizers means that they will have temporal selectivity and be taken up more slowly by the healthy human cells, than by the bacterial cells.

Potential dyes and photosensitizers that may be used in various embodiments, and their absorption spectra wavelengths for photochemical interactions, include but are not limited to the following:

| | Spectral Absorption |
|---|---|
| | Ranges and Peaks |
| Photosensitizer | (nm) |
| Acid Fuchsin | 530-560 |
| ALA | 635 |
| ALA esters | 635 |
| Allura Red AC | 504 |
| Aniline Blue | 550-620 |
| Aramath | 523 |
| Azure B | 580+ |
| Azure C | 580-640 |
| Basic Fuchsin | 520-570 |
| Benzoporphyrine derivative | 400, 585, 687 |
| mono-acid | |
| Beta carotene | 470 |
| BPD-ma | 689 |
| Brilliant Cresyl Blue | 550+ |
| Brilliant Scarlet 4R | 503 |
| Carmine | 500-570 |
| Carmoisine azorubine | 515 |
| Ce6-PVP (Fotolon) Ce6 | 660 |
| derivatives (Radachlorin, | |
| Photodithazine) | |
| Chlorin e6 | 410, 658 |
| Chlorophylls and copper | 633 |
| complexes | |
| Chromotrope FB | 512 |
| Cochineal | 530 |
| Congo Red | 400-560 |
| Crystal Violet | 550-610 |
| Curcumin | 435 |
| Darrow Red | 450-550 |
| Eosin Y | 490-530 |
| Erioglaucine disodium salt | 629 |
| Erythrosin B | 510-540 |
| Ethyl Eosin | 530-550 |
| Fast Green | 560+ |
| Giemsa | 500+ |
| Hematoporphyrine derivative | 505, 537, 565 |
| HPDprofimer sodium | 630 |
| HPPH | 665 |
| hypericin | 550, 595 |
| Indigo Carmine | 608 |
| Light Green SF | 590+ |
| Lissamine green | 633 |
| Lutetium texaphyrin | 470, 730 |
| Luxol Fast Blue | 500-640 |
| Lycopene | 530 |
| Methyl Green | 560+ |
| Methylene Blue | 590+ |
| Mono-N-Aspartyl Chlorin e6 | 664 |
| m-THPC | 652 |
| Neutral Red | 480-570 |
| New Coccine | 506 |
| Nickel 5,10-bis-acrylate | 580 |
| etioporphyrin I | |
| Nigrosin | 450+ |
| Nuclear Fast Red | 460-550 |
| Orange G | 450-510 |
| Orcein | 500-620 |
| Profimer Sodium Photofrine | 630 |
| (HPD) | |
| Phloxine B | 520+ |
| Phthalocyanine-4 | 670 |
| Protoporphyrin IX | 506, 546, 578, 563 |
| Prussian Blue | 560+ |
| Pyronin B | 510-560 |
| Rose bengal | 548 |
| Riboflavin 5′-monophosphate | 441 |
| sodium salt | |
| Saffron | 350-480 |
| Safranin O | 470-550 |
| Silicon phthalocyanine (Pc4) | 675 |
| SnEt2 (Purlytin) | 660 |
| Sudan IV | 470-580 |
| Sudan Red | 450-590 |
| Talaporfin (LS11, MACE, | 660 |
| Npe6) | |
| Taporfin Sodium | 664 |
| Tartrazine | 400-460 |
| Tin etiopurpurin | 660 |
| Temoporfin (Foscan) (mTHPC) | 652 |
| TiO₂ Anatase P25 Degussa | 525-578 |
| Toluidine Blue | 560+ |
| Trypan Blue | 500+ |
| Verteporfin | 690 |
| 5,10-octaethylbacteriopurpurin | 563, 598 |
| 5,15-octaethylbacteriopurpurin | 558, 592 |

The various embodiments may be implemented using a number of techniques and/or materials, which may be selected to optimize effects based on the particular targeted pathogen.

In some examples, a photosensitizer may be chosen that demonstrates (a) preferential absorption by the desired target pathogenic bacteria over healthy lung cells and tissues and (b) maximum absorption of light at wavelengths corresponding to the visible spectral peak wavelengths of light generated by a therapeutic bioluminescent bacterial species. In various embodiments, the photosensitizer may be delivered to the lungs through any number of routes of administration (e.g., via inhalation, oral administration, sublingual administration, buccal administration, intravenous injection, subcutaneous administration, etc.). A route of administration for the photosensitizer may be selected based on a number of factors, but with the overall aim of safely and evenly distributing the photosensitizer throughout the lung alveoli. In the various embodiments, once distributed in the lung alveoli, adequate waiting time may be required to allow the photosensitizer to penetrate the pathogenic bacteria of a lung infection (e.g. Pseudomonas or Mycobacteria lesion). In the various embodiments, the ratio of absorption of the photosensitizer by pathogenic bacteria compared to the absorption by normal tissue cells may be relatively high.

In various embodiments, following the waiting time for the photosensitizer, the bioluminescent bacteria may be delivered to the lungs. Similar to the photosensitizer, such delivery may be performed through any of a number of routes of administration (e.g., via inhalation). The route of administration for the bioluminescent bacteria may be selected based on a number of factors, but with the overall aim of safely and evenly distributing the bacteria throughout the lung alveoli.

In various embodiments, a device or component of a device may be used to introduce the bioluminescent bacteria to the lungs. An example of such device (or “introducer”) may be part of an inhaler or similar apparatus, and may be used additionally or alternatively to administer the photosensitizer as discussed above. The user may take a slow breath inward as the inhaler delivers a dose, hold the breath for several seconds, and repeat as necessary. In an example embodiment, a spacer may be attached to the inhaler to makes it easier to put the bioluminescent bacteria into a holding chamber so that the inhalation will have maximum availability to the lungs.

Once distributed, the bioluminescent bacteria may emit light having particular wavelengths in the visible spectral range, which may illuminate/excite the photosensitizer to generate ROS. In various embodiments, such ROS may reduce or kill the pathogenic bacteria causing infection in the lung.

In some embodiments, an antibiotic agent may be administered following the PDT treatment (i.e., after the bioluminescent bacteria have activated the photosensitizer). In some embodiments, an antifungal agent may be administered following the PDT treatment (i.e., after the bioluminescent bacteria have activated the photosensitizer and weakened pathogenic fungi). Administration of the antibiotic or antifungal agent may be performed via any suitable route (e.g., via inhalation, oral administration, sublingual administration, buccal administration, intravenous injection, subcutaneous injection, etc.), depending on the particular formulation being used. In various embodiments, the antibiotic or antifungal agent may assist in killing the pathogen that has been weakened by the PDT treatment. In some embodiments, the device or apparatus used to introduce the bioluminescent bacteria to the lungs may be used to administer the antibiotic or antifungal agent. In some embodiments, different equipment may be used for the PDT treatment and administering the antibiotic or antifungal agent.

While the various embodiments are described herein with respect to targeting pathogenic bacteria, those of ordinary skill in the art will recognize that the pulmonary PDT treatments of the various embodiments may be applicable to other harmful microscopic pathogens that are susceptible to damage from reactive oxygen species. Examples of such other microscopic pathogens may include other microbes (e.g., archaea, fungi, protozoa) and/or non-living pathogens (e.g., viruses, viroids, etc.). Further, while the various embodiments are described herein with respect to human lungs, the lung is provided merely as an example organ in which the various embodiments may be implemented. However, embodiment PDT treatments may be applicable to many other human organs and/or systems, as well as to various animal organs and systems.

In general, it is to be understood that the technology described herein may be used in conjunction with any of the devices, systems, techniques, etc. that may be known to those of ordinary skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used.

Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for treating a pathogenic infection in a human or animal subject, comprising:
performing photodynamic therapy on a lung of the subject, wherein performing the photodynamic therapy comprises:
introducing a photosensitizer into the lung of the subject; and
introducing bioluminescent bacteria into the lung of the subject, wherein treatment of the pathogenic infection is initiated by light emitted from the bioluminescent bacteria.

2. The method of claim 1, wherein the light emitted from the bioluminescent bacteria activates or potentiates the photosensitizer.

3. The method of claim 1, wherein the light emitted from the bioluminescent bacteria is emitted in a selected wavelength range.

4. The method of claim 1, wherein the bioluminescent bacteria is compatible with the commensal microbiome of the lung.

5. The method of claim 1, wherein the photosensitizer comprises a dye.

6. The method of claim 1, wherein treating the pathogenic infection comprises treating one or more pathogenic bacterial species.

7. The method of claim 1, wherein treating the pathogenic infection comprises treating one or more pathogenic fungal species.

8. The method of claim 6, further comprising:
administering an antibiotic agent after performing the photodynamic therapy, wherein the antibiotic agent is capable of killing weakened pathogenic bacteria.

9. The method of claim 7, further comprising:
administering an antifungal agent after performing the photodynamic therapy, wherein the antifungal agent is capable of killing weakened pathogenic fungi.

10. The method of claim 1, wherein introducing the bioluminescent bacteria into the lung of the subject comprises introducing a phototherapeutically effective concentration of bioluminescent bacteria throughout a substantial portion of an interior surface area of the lung.

11. The method of claim 10, wherein the substantial portion of the interior surface area of the lung comprises an area of at least about 50 m².

12. The method of claim 10, wherein the substantial portion of the interior surface area of the lung comprises an area of at least about 10 m².

13. The method of claim 10, wherein the substantial portion of the interior surface area of the lung comprises an area of at least about 70 m².

14. A device comprising:
a reservoir containing bioluminescent bacteria; and
an introducer configured to introduce the bioluminescent bacteria into a lung of a human or animal subject in an amount sufficient to perform photodynamic therapy on the lung using light emitted from the bioluminescent bacteria.

15. The device of claim 14, further comprising a reservoir containing a photosensitizer, wherein the introducer is configured to introduce the photosensitizer to the lung, and wherein the photosensitizer is configured to be activated or potentiated by light emitted from the bioluminescent bacteria.

16. The device of claim 14, wherein the introducer comprises an inhaler device.

17. The device of claim 15, wherein the photosensitizer is preferentially absorbed by a target pathogen over healthy lung tissue, and wherein the photosensitizer maximally absorbs light at wavelengths in the visible spectral range corresponding to the light emitted from the bioluminescent bacteria.

18. The device of claim 16, wherein the introducer is configured to introduce the bioluminescent bacteria into the lung after expiration of a predetermined first time period following introduction of the photosensitizer.

19. The device of claim 17, wherein the device is further configured to administer an antibiotic or antifungal composition to the subject after expiration of a predetermined second time period following introduction of the photosensitizer, wherein the predetermined second time period is of sufficient duration to allow activation of the photosensitizer.

20. The device of claim 19, wherein the photodynamic therapy weakens the target pathogen, and wherein the antibiotic or antifungal composition is capable of killing the weakened target pathogen.
